(19) United States
(12) Patent Application Publication
(10) Pub. No.: US 2022/0034898 A1
(43) Pub. Date: Feb. 3, 2022

(54) DETERMINING FETAL LUNG MATURITY USING A MATERNAL SAMPLE

(71) Applicant: Robert A. WELCH, Brooklyn, MI (US)

(72) Inventor: Robert A. WELCH, Brooklyn, MI (US)

(21) Appl. No.: 17/384,650

(22) Filed: Jul. 23, 2021

Related U.S. Application Data
(60) Provisional application No. 63/058,412, filed on Jul. 29, 2020.

(51) Int. Cl. G01N 33/68

(52) U.S. Cl. CPC G01N 33/689; G01N 2333/91057; G01N 2800/36

(57) ABSTRACT

A method of determining fetal lung development, by taking a sample from a pregnant subject, applying the sample to a panel including at least one biomarker for fetal lung maturity, measuring a response of the sample to the biomarker, and determining fetal lung maturity. A panel including an assay with at least one biomarker for fetal lung maturity on a solid support.

### BACKGROUND OF THE INVENTION

### 1. Technical Field

The present invention relates to compositions and methods for determining fetal lung maturity. More specifically, the present invention relates to assays for determining fetal lung maturity from maternal blood.

### 2. Background Art

The ability to breath air is an essential physiologic process innate to the vast majority of healthy term newborns. This process is accomplished through the production of surfactant, which coats the internal lining of the lungs to prevent them from collapsing between breaths. This reduces the work of breathing and allows for comfortable respiration without exhaustion. Pneumocytes, or alveolar cells, are the cells that line the alveoli (the tiny air sacs in the lungs that allow for rapid gaseous exchange) and comprise of the majority of the inner surface of the lungs.

There are two types of alveolar cells—type I pneumocytes and type II pneumocytes. Type I pneumocytes are involved in the process of gas exchange between the alveoli and the capillaries. They are squamous (flattened) in shape and extremely thin (˜0.15 μm)—minimizing diffusion distance for respiratory gases. Type I pneumocytes are connected by occluding junctions, which prevents the leakage of tissue fluid into the alveolar air space. They are amitotic and unable to replicate however type II cells can differentiate into type I cells if required. Type II pneumocytes are responsible for the secretion of pulmonary surfactant, which reduces surface tension in the alveoli. They are cuboidal in shape and possess many granules (for storing surfactant components). Type II pneumocytes only comprise a fraction of the alveolar surface (˜5%) but are relatively numerous (˜60% of total cells). The surfactant they produce is a biochemical complex made up mostly of phosphatidylcholine and phosphatidylglycerol. These are synthesized by lysophosphatidylcholine acyltransferase 1 (LPCAT 1) (Harayama, et al., Eliis, et al.).

Surfactant production peaks by 40 weeks with virtually no normal newborns developing respiratory distress syndrome (RDS). However, up to 2% of babies born at 36 weeks develop RDS and 8-23% of those born at 34 weeks develop RDS. Essentially all newborns at 30 weeks or less have immature lungs and will develop some expression of RDS. Gender and ethnicity contribute to lung maturity in an unpredictable fashion.

This variation in fetal lung production of surfactant remains a therapeutic dilemma when obstetricians and midwives make decisions about timing of delivery in several conditions arising during pregnancy. These include maternal hypertension, preeclampsia, HELLP syndrome, premature rupture of the amniotic membranes, intrauterine growth restriction, maternal smoking or illicit drug use, maternal hemoglobinopathies, and diabetes. Treatment of premature labor is often prescribed without knowledge of the maturity of the fetal lungs. Further, because perinatal practitioners do not know the fetal lung maturity status, pregnant women are often transferred significant distances from a hospital with lesser newborn resuscitation and care capabilities to one that is considered tertiary care. Newborns delivered before their gestational term are considered premature and often have respiratory difficulty. The development of respiratory distress syndrome can lead to a cascade of adverse sequalae, including neonatal asphyxia, necrotizing enterocolitis, intracranial hemorrhage, cerebral palsy and death. The premature newborn lacks physiologic maturity leading to an inability to produce adequate amounts of lung surfactant.

In an attempt to cause fetal lungs to mature, physicians administer glucocorticoids to mothers anticipating a preterm birth (for almost any obstetrical diagnosis) in an attempt to reduce the severity of newborn RDS. A single course of corticosteroids is often prescribed between 24 0/7 and 36 6/7 weeks of gestation (Management of preterm labor. Practice Bulletin No. 171. American College of Obstetricians and Gynecologists. Obstet Gynecol 2016; 128:e155-64, and Roberts, et al.). Unfortunately, corticosteroid administration may increase maternal morbidities (e.g. difficult blood sugar control in diabetics). Multiple repeated courses of corticosteroids are also concerning for the fetus since the medication is meant to cross the placenta to provide fetal therapy. In particular, some studies suggest decreased fetal brain growth, possible deleterious effects on cerebral myelination, and other concerns (Roberts, et al.) with corticosteroid treatment. It is also probable that patients between 32-36 6/7 weeks range are carrying fetuses with lungs that are already producing enough surfactant to be mature. These patients are getting unnecessary corticosteroids. So, knowing the maturity of the fetal lungs is an integral piece of knowledge essential for the practice of perinatal medicine. (Towers, et al.)

Prior art solutions have focused on developing fetal lung maturity tests by measuring phospholipids and lamellar bodies in maternal amniotic fluid. While several of these tests are beneficial, they all require a procedure known as amniocentesis. During this procedure, the perinatal specialist introduces a needle into the maternal abdomen under ultrasound guidance. The amniotic fluid retrieved is then subjected to studies to predict the state of fetal lung maturity. Unfortunately, this procedure is uncomfortable for the mother and is a difficult concept for many to contemplate and give consent. Further, it requires an expert perinatal specialist with great experience in doing the procedure. Since few are performed currently, these experts are difficult to find. Even when an expert is available, there has to be a large enough pocket of amniotic fluid to retrieve a sample. Finally, even in expert hands, a small number of patients' experience complications from bleeding if the placenta is penetrated or the umbilical cord is pierced. Premature labor and delivery may result from the procedure. While generally a safe procedure, a small number of stillbirths are related annually to amniocentesis.

There remains a need for a method of accurately assessing fetal lung development without invasive procedures such as amniocentesis.

### SUMMARY OF THE INVENTION

The present invention provides for a method of determining fetal lung development, by taking a sample from a pregnant subject, applying the sample to a panel including at least one biomarker for fetal lung maturity, measuring a response of the sample to the biomarker, and determining fetal lung maturity.

The present invention also provides for a panel including an assay with at least one biomarker for fetal lung maturity on a solid support.

### DETAILED DESCRIPTION OF THE INVENTION

Generally, the present invention provides for methods and assays for determining fetal lung maturity and development without the need for invasive amniocentesis in the pregnant subject. More specifically, the present invention provides for a method of determining fetal lung development, by taking a sample from a pregnant subject, applying the sample to a panel including at least one biomarker for fetal lung maturity, measuring a response of the sample to the biomarker, and determining fetal lung maturity. The present invention also provides for a panel including an assay with at least one biomarker for fetal lung maturity on a solid support.

“Pregnant subject” as used herein, refers to any pregnant human or animal.

“Sample” as used herein, refers to any type of sample taken from the pregnant subject, including, but not limited to, blood, plasma, serum, urine, saliva, nasal fluid, or any other fluid. The sample can be processed as necessary to apply to the panel, such as by centrifugation.

“Panel” as used herein, refers to an immunoassay assay on a solid support, such as, but not limited to an ELISA (such as sandwich ELISA), radioimmunoassay, real-time immunoquantitative PCR, protein microarrays, or electrochemiluminescent assays. The ELISA panel can use a single threshold well. Results on the panel can be read with colorimetry or by visual analysis, i.e. a result of fetal lung maturity can be one color, and immaturity can be a different color.

The biomarker for fetal lung maturity is preferably anti-LPCAT1 antibodies. The biomarker can also be anti-LPCAT2 antibodies, anti-LPCAT3 antibodies, or anti-LPCAT4 antibodies. Any other biomarker that provides a measure of fetal lung maturity can also be used. The panel can include combinations of the biomarkers. The biomarkers can be obtained from humans, non-human species, or bacteria.

Through the use of RT-PCR, it has previously been established that LPCAT1 mRNA in amniotic fluid and maternal plasma correlates with the lamellar body count (LBC) in the amniotic fluid. (Welch, et al. 2016, Welch, et al. 2018) The LBC is a well-established clinical marker of fetal lung maturity. Using some of Welch's samples, the maternal plasma LPCAT1 protein has also recently been measured using ELISA (Aras, et al.). Maternal plasma can be acquired by venipuncture which generally consists of drawing blood from the pregnant subject's arm often along with other routine clinical laboratory studies. Other than occasionally causing a bruise at the venipuncture site, this approach is far better tolerated. Maternal plasma is then prepared from the sample using simple centrifugation.

Using maternal plasma, ELISA can be used to quantify LPCAT1 protein related to newborn clinical outcomes and need for respiratory support. A threshold number of anti-LPCAT1 antibodies is associated with minimal need or no need for newborn respiratory support. Further, this threshold number of anti-LPCAT1 antibodies can be affixed into an ELISA well. By taking maternal blood, centrifuging the blood to produce plasma, then putting the plasma into a well in a panel while performing an ELISA assay, it can be determined whether the sample meets a colorimetric (or visual) level corresponding to whether the fetal lungs are mature, or not. This procedure does not require invasive testing (i.e., amniocentesis). It also avoids the multiple dilution approach used by traditional ELISA in that the technique uses a “threshold well” containing a preset number of anti-LPCAT1 antibodies corresponding to the number needed to predict fetal lung maturity.

Depending on the results of the assay, it can be determined if the fetal lungs are mature and medical decisions can be further made for the pregnant subject and baby by a doctor or medical professional. For example, if the fetal lungs are mature, it can be decided to keep the pregnant subject at a medical site and not transfer to a larger hospital that may be further away, putting the pregnant subject at risk. If the fetal lungs are mature, it can be decided to not treat the pregnant subject with certain medication (such as corticosteroids). If the fetal lungs are mature, it can be decided that it is safe to deliver the baby early.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### REFERENCES

1. Harayama T, Shindou H, Shimizu T., Biosynthesis of phosphatidylcholine by human lysophosphatidylcholine acyltransferasel. J Lipid Res. 2009 Sep; 50(9):1824-31.
2. Ellis B, Kaercher L, Snavely C, Zhao Y, Zou C., Lipopolysaccharide triggers nuclear import of Lpcat1 to regulate inducible gene expression in lung epithelia., World J Biol Chem. 2012 Jul 26; 3(7):159-66.
3. Management of preterm labor. Practice Bulletin No. 171. American College of Obstetricians and Gynecologists. Obstet Gynecol 2016; 128:e155-64.
4. Roberts D, Dalziel S R. Antenatal corticosteroids for accelerating fetal lung maturation for women at risk of preterm birth. Cochrane Database of Systematic Reviews 2006, Issue 3. Art. No.: CD004454. DOI: 0.1002/14651858.CD004454.pub2.
5. Towers C V, Freeman R K, Nageotte M P, Garite T J, Lewis D F, Quilligan E J. The case for amniocentesis for fetal lung maturity in late-preterm and early-term gestations. Am J Obstet Gynecol. 2014 Feb; 210(2):95-6. Epub 2013 Oct 15.
6. Welch R A, Shaw M K, Welch KC, Amniotic fluid LPCAT1 mRNA correlates with the lamellar body count, J. Perinatal Med, 2016 Jul 1; 44(5):531-23.
7. Welch R A, Recanati M A, Welch KC, Shaw M K, Maternal Plasma LPCAT1 mRNA correlates with the lamellar body count, J Perinatal Med, 2018 May 24; 46(4):429-431. Doi: 10.1515/jpm-2017-0057.
8. Aras S; Minchella, Paige, Welch, K C; Patek, K D, Welch, R A; Recanati, M A. Maternal Plasma Lysophospholipid Acyltransferase 1 Protein Levels Correlate With Fetal Lung Maturity [31F] Obstet & Gynecol: May 2019—Volume 133—Issue-p 70S doi: 10.1097/01.ACOG.0000559062.10510.aa

What is claimed is:

1. A method of determining fetal lung development, including the steps of:
taking a sample from a pregnant subject;
applying the sample to a panel including at least one biomarker for fetal lung maturity;
measuring a response of the sample to the biomarker; and
determining fetal lung maturity.

2. The method of claim 1, wherein the sample is chosen from the group consisting of blood, plasma, serum, urine, saliva, and nasal fluid.

3. The method of claim 1, wherein the panel is further defined as an immunoassay on a solid support chosen from the group consisting of ELISA, radioimmunoassay, real-time immunoquantitative PCR, protein microarrays, and electrochemiluminescent assays.

4. The method of claim 1, wherein said measuring step further includes the step of reading the panel with colorimetry.

5. The method of claim 1, wherein the biomarker is chosen from the group consisting of anti-LPCAT1 antibodies, anti-LPCAT2 antibodies, anti-LPCAT3 antibodies, anti-LPCAT4 antibodies, and combinations thereof.

6. The method of claim 1, wherein the panel includes a well having a threshold number of biomarkers affixed therein indicating minimal need or no need for newborn respiratory support and said measuring step further includes comparing a colorimetric value of the sample to the threshold well to determine fetal lung maturity.

7. The method of claim 1, further including the step of a doctor performing a medical decision about the pregnant subject based on the results of said determining step.

8. The method of claim 7, wherein the fetal lungs are determined to be mature and the medical decision is chosen from the group consisting of keeping the pregnant subject at a medical site, not treating the pregnant subject with medication, and delivering a baby early.

9. A panel comprising an assay with at least one biomarker for fetal lung maturity on a solid support.

10. The panel of claim 9, wherein said assay is chosen from the group consisting of ELISA, radioimmunoassay, real-time immunoquantitative PCR, protein microarrays, and electrochemiluminescent assays.

11. The panel of claim 9, wherein said at least one biomarker is chosen from the group consisting of anti-LPCAT1 antibodies, anti-LPCAT2 antibodies, anti-LPCAT3 antibodies, anti-LPCAT4 antibodies, and combinations thereof.

12. The panel of claim 9, wherein said panel includes a well having a threshold number of biomarkers affixed therein indicating minimal need or no need for newborn respiratory support and comparable to a sample with colorimetry.

13. The panel of claim 9, wherein said panel is configured to receive a sample chosen from the group consisting of blood, plasma, serum, urine, saliva, and nasal fluid.
